(19) United States
(12) Patent Application Publication
(10) Pub. No.: US 2023/0268073 A1
(43) Pub. Date: Aug. 24, 2023

(54) INQUIRY INFORMATION PROCESSING METHOD AND APPARATUS, AND MEDIUM

(71) Applicant: BEIJING SOGOU TECHNOLOGY DEVELOPMENT CO., LTD., Beijing (CN)

(72) Inventor: Junqing HE, Beijing (CN)

(21) Appl. No.: 18/137,960

(22) Filed: Apr. 21, 2023

Related U.S. Application Data
(63) Continuation of application No. PCT/CN2021/103667, filed on Jun. 30, 2021.

(30) Foreign Application Priority Data
Jan. 26, 2021 (CN) 202110105880.9

(51) Int. Cl. G16H 50/20

(52) U.S. Cl. CPC G16H 50/20

(57) ABSTRACT

Embodiments of this application provide an inquiry information processing method performed at a computing device. The method specifically includes: determining user disease features according to at least one user input; performing disease prediction on the user disease features to obtain corresponding candidate diseases; acquiring question entities from a knowledge graph according to disease feature entities corresponding to the candidate diseases, the question entities being used for representing questions related to the disease feature entities; and generating target questions according to the question entities, the target questions being used for performing an inquiry on a user. By means of the embodiments of this application, the accuracy of target questions for an inquiry can be improved, thereby improving the inquiry efficiency.

### CROSS-REFERENCE TO RELATED APPLICATIONS

This application is a continuation application of PCT Patent Application No. PCT/CN2021/103667, entitled “INQUIRY INFORMATION PROCESSING METHOD AND APPARATUS, AND MEDIUM” filed on Jun. 30, 2021, which claims priority to Chinese Patent Application No. 202110105880.9, entitled “INQUIRY INFORMATION PROCESSING METHOD AND APPARATUS, AND MEDIUM” filed with the China National Intellectual Property Administration on Jan. 26, 2021, all of which are incorporated herein by reference in their entirety.

### FIELD OF THE TECHNOLOGY

Embodiments of this application relate to the field of medical technologies, and in particular, to an inquiry information processing method and apparatus, and a medium.

### BACKGROUND OF THE DISCLOSURE

Inquiry is a method for diagnosing diseases by purposely inquiring patients or surrogates to understand the occurrence, development, diagnosis and treatment process, and current symptoms of the diseases, and all other conditions related to the diseases. With the continuous development of an artificial intelligence technology, the way of an artificial intelligence-based inquiry has gradually developed, which brings a lot of convenience to the life of users.

In the current inquiry information processing method, a question and answer pair is usually pre-configured. In this way, when inquiry data input by a user is received, target answer data matching the inquiry data is queried from the pre-configured question and answer pair, and the queried target answer data is fed back to the user.

In practical applications, when target answer data matching the inquiry data is not queried, a manual inquiry procedure needs to be triggered for the inquiry data, so as to manually answer the inquiry data. Therefore, the current inquiry information processing method has a problem of low inquiry efficiency.

### SUMMARY

Embodiments of this application provide an inquiry information processing method and apparatus, and a medium, which can improve the accuracy of target questions for an inquiry, thereby improving the inquiry efficiency.

To resolve the foregoing problems, embodiments of this application disclose an inquiry information processing method performed at a computing device, the method including:
determining user disease features according to at least one user input;
performing disease prediction on the user disease features to obtain corresponding candidate diseases;
acquiring question entities from a knowledge graph according to disease feature entities corresponding to the candidate diseases, the question entities being used for representing questions related to the disease feature entities; and
generating target questions according to the question entities, the target questions being used for performing an inquiry on a user.

According to another aspect, embodiments of this application disclose an inquiry information processing apparatus, including:
a user disease feature determination module, configured to determine user disease features according to at least one user input;
a user disease feature processing module, configured to perform disease prediction on the user disease features to obtain corresponding candidate diseases;
a question entity acquisition module, configured to acquire question entities from a knowledge graph according to disease feature entities corresponding to the candidate diseases, the question entities being used for representing questions related to the disease feature entities; and
a first question generation module, configured to generate target questions according to the question entities, the target questions being used for performing an inquiry on a user.

According to yet another aspect, embodiments of this application disclose an apparatus for processing inquiry information, including a memory, and one or more programs. The one or more programs are stored in the memory and implement, when executed by one or more processors, the steps of the foregoing method.

According to yet another aspect, embodiments of this application disclose a non-transitory computer-readable medium, having stored thereon instructions which, when executed by one or more processors, enable an apparatus to perform the inquiry information processing method as described in one or more of the foregoing.

The embodiments of this application include the following advantages:

In the embodiments of this application, disease prediction processing is dynamically performed based on user disease features determined by at least one user input by processing inquiry information during an inquiry, and target questions are dynamically generated. Since the target questions for the inquiry are automatically generated by processing the inquiry information during the inquiry in the embodiments of this application, the inquiry efficiency can be improved.

Furthermore, in the embodiments of this application, the process of performing disease prediction on the inquiry information and generating target questions may be a dynamic process. Therefore, the target questions more related to the user disease features can be obtained according to the accumulation of the user disease features in the inquiry process, and the rationality of the inquiry according to the target questions can thus be improved. Moreover, candidate diseases more matching the user disease features can be obtained according to the accumulation of the user disease features in the inquiry process, and the accuracy of generating target questions for disease prediction processing can thus be improved. In addition, in the embodiments of this application, it may be dynamically determined whether the inquiry information processing stops in advance according to the predicted disease features, the user disease features, number of inquiry rounds, and other information, so as to improve the inquiry efficiency, thereby improving the user experience.

### BRIEF DESCRIPTION OF THE DRAWINGS

To describe the technical solutions in embodiments of this application more clearly, the following briefly describes the accompanying drawings required for describing the embodiments of this application. Apparently, the accompanying drawings in the following description show merely some embodiments of this application, and those of ordinary skill in the art may derive other drawings from these accompanying drawings without creative efforts.

FIG. 1 is a schematic diagram of an application environment of an inquiry information processing method according to an embodiment of this application.

FIG. 2 is a flowchart of steps in Embodiment 1 of an inquiry information processing method according to this application.

FIG. 3 is a schematic diagram of a disease entity and attributes thereof according to an embodiment of this application.

FIG. 4 is a schematic diagram of a disease feature entity and attributes thereof according to an embodiment of this application.

FIG. 5 is a flowchart of steps in Embodiment 1 of a knowledge graph processing method according to this application.

FIG. 6 is a flowchart of steps in Embodiment 4 of an inquiry information processing method according to this application.

FIG. 7 is a flowchart of steps in Embodiment 6 of an inquiry information processing method according to this application.

FIG. 8 is a flowchart of steps in Embodiment 7 of an inquiry information processing method according to this application.

FIG. 9 is a flowchart of steps in Embodiment 8 of an inquiry information processing method according to this application.

FIG. 10 is a structural block diagram of an embodiment of an inquiry information processing apparatus according to this application.

FIG. 11 is a block diagram of an apparatus 1100 for processing inquiry information according to this application.

FIG. 12 is a schematic structural diagram of a server according to some embodiments of this application.

### DESCRIPTION OF EMBODIMENTS

The following clearly and completely describes the technical solutions in the embodiments of this application with reference to the accompanying drawings in the embodiments of this application. Apparently, the described embodiments are some embodiments of this application rather than all of the embodiments. All other embodiments obtained by those of ordinary skill in the art based on the embodiments of this application without creative efforts shall fall within the protection scope of this application.

In view of the technical problem of low inquiry efficiency in the conventional technology, embodiments of this application provide an inquiry information processing method. The method may include: determining user disease features according to at least one user input; performing disease prediction on the user disease features to obtain corresponding candidate diseases; and generating target questions according to disease features corresponding to the candidate diseases, the target questions being used for performing an inquiry on a user.

In the embodiments of this application, at least one user input of a user may include: disease features of the user (hereinafter referred to as user disease features). In the embodiments of this application, the user disease features may be determined based on an inquiry and processed. Types of the disease features may include: symptoms, incentives, high incidence seasons, contact history, family history, etc.

In the embodiments of this application, disease prediction processing is dynamically performed based on user disease features obtained by at least one user input by processing inquiry information during an inquiry, and target questions are dynamically generated. Since the target questions for the inquiry are automatically generated by processing the inquiry information during the inquiry in the embodiments of this application, the inquiry efficiency can be improved.

Furthermore, in the embodiments of this application, the process of performing disease prediction on the inquiry information and generating target questions may be a dynamic process. Therefore, the target questions more related to the user disease features can be obtained according to the accumulation of the user disease features in the inquiry process, and the rationality of the inquiry information processing can thus be improved. Moreover, candidate diseases more matching the user disease features can be obtained according to the accumulation of the user disease features in the inquiry process, and the accuracy of generating target questions for disease prediction processing can thus be improved.

The inquiry information processing method provided by the embodiments of this application may be applied to an application scenario such as a website and/or an application (APP). For example, the application scenario of the embodiments of this application may include: medical related websites, or medical related APPs, etc.

The inquiry information processing method provided by the embodiments of this application may be applied in an application environment shown in FIG. 1. As shown in FIG. 1, a client 100 and a server 200 are located in a wired or wireless network. The client 100 and the server 200 perform data interaction through the wired or wireless network.

In some embodiments, the client 100 may run on a terminal, and the terminal may include, but not limited to: a smart mobile phone, a tablet computer, an e-book reader, a moving picture experts group audio layer III (MP3) player, a moving picture experts group audio layer IV (MP4) player, a laptop portable computer, a vehicle-mounted computer, a desktop computer, a set-top box, a smart television, a wearable device, and the like.

In practical applications, the client 100 may interact with a user. Specifically, the client 100 may receive at least one user input and provide target questions to the user.

The client 100 may generate the target questions using the inquiry information processing method according to the embodiments of this application. Or, the client 100 may transmit at least one user input of the user to the server 200, whereby the server 200 generates target questions for an inquiry using the inquiry information processing method according to the embodiments of this application.

### Method Embodiment 1

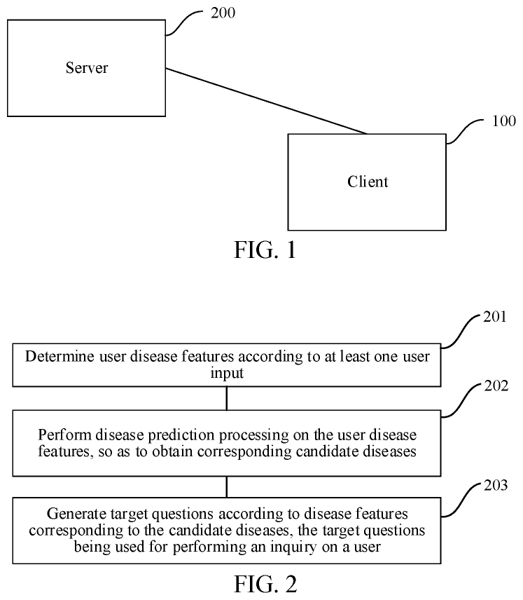

Referring to FIG. 2, a flowchart of steps in Embodiment 1 of an inquiry information processing method according to this application is shown. The method may specifically include the following steps:

Step 201: Determine user disease features according to at least one user input.

Step 202: Perform disease prediction on the user disease features to obtain corresponding candidate diseases.

Step 203: Generate target questions according to disease features corresponding to the candidate diseases, the target questions being used for performing an inquiry on a user.

At least one of the steps of the method embodiment shown in FIG. 2 may be executed by a client and/or a server, although the embodiments of this application are not limited to the particular body executing the various steps.

In step 201, at least one user input may be received by means of keyboard input, option selection, voice input, and other input manners.

In some embodiments, the at least one user input may include:
an active input; or
an active input and a reply to preset questions; or
an active input and a reply to the target questions; or
an active input and replies to preset questions and target questions. The reply may include text input, selection of answer options, etc. without limiting the form of the reply.

The preset questions may be pre-stored questions relative to target questions dynamically generated according to the at least one user input.

For example, in the inquiry procedure, the first input by the user is typically an active input. The active input typically includes: a chief complaint. In the field of medical technology, the chief complaint is used for representing narration of a patient or a surrogate for the most prominent symptoms and/or signs. The chief complaint typically includes: at least one of the symptoms and signs stated by the patient or the surrogate, nature, and duration.

Upon receiving the active input, the user may be provided with preset questions, so as to acquire a reply from the user to the preset questions.

The preset questions may be questions of which the frequency exceeds a frequency threshold in the inquiry. For example, keywords of the preset questions may include: duration, mental state, etc., so as to inquire the duration of symptoms and the mental state of the patient. It is to be understood that the embodiments of this application are not limited to the specific preset questions.

In some embodiments, the target questions specifically include question text and answer options, and then the at least one user input specifically includes answer options selected by the user.

Examples of the question text may include: “Did you vomit directly without nausea?”, “What are the symptoms?”, “What diseases did you have before?”, “What is the type of rash?”, “What is the shape of stool”, etc. The answer options are used for representing available answer options.

For yes-or-no questions such as “Did you vomit directly without nausea?”, the answer options may include: [Yes, No, I don't know], etc.

For non-yes-or-no questions such as “What are the symptoms?”, the answer options may include: answer options corresponding to disease features. For example, the answer options may include: [symptom 1, symptom 2, . . . , symptom N, none of the foregoing symptoms].

In an optional embodiment of this application, user disease features may be determined from at least one user input. Methods for determining the user disease features may include, but not limited to: an entity identification method, or a matching method of disease feature tables, etc. The determined user disease features may be saved to a user disease feature set.

It is to be noted that the at least one user input may include a standard description corresponding to the user disease features. Or, the at least one user input may include a non-standard description corresponding to the user disease features, such as a colloquial description. In this case, the non-standard description in the at least one user input may be converted into a standard description. Therefore, in the embodiments of this application, the standard description of the user disease features will be used for performing disease prediction on the standardized user disease features, so as to improve the accuracy of disease prediction.

In the embodiments of this application, disease features corresponding to diseases may be determined according to medical resources such as medical books, medical databases and medical question and answer data; or, a knowledge graph may be constructed to determine the disease features corresponding to the diseases. The relevant content of the knowledge graph will be described in the subsequent embodiments.

In step 202, the disease prediction processing may be used for determining probabilities of candidate diseases corresponding to the user disease features. There may be at least one candidate disease which may correspond to a score that may represent a probability of the candidate disease under the condition of the user disease features.

The foregoing operation of performing disease prediction on the user disease features may specifically include: determining candidate diseases corresponding to the user disease features according to matching information between the user disease features and disease features corresponding to diseases. For example, if the user disease features match disease features of disease A, disease A may be taken as a candidate disease corresponding to the user disease features.

In the embodiments of this application, the process of disease prediction on the user disease features may be a dynamic process. When a user disease feature set is updated, disease prediction processing may be performed on the updated user disease feature set. In this way, candidate diseases more matching the user disease features can be obtained according to the accumulation of the user disease features in the inquiry process, and the accuracy of target questions for the inquiry generated accordingly can thus be improved.

In step 203, the target questions may be used for performing an inquiry on the disease features corresponding to the candidate diseases, so as to assist the user in determining whether corresponding disease features are present.

In some embodiments, the target questions may include: question text and answer options. The answer options correspond to the corresponding disease features of the candidate diseases. In this way, it may be determined whether the user has corresponding disease features according to a selection operation of the user for the answer options. When it is determined that the user has corresponding disease features, the answer options selected by the user may be converted into the corresponding user disease features in the embodiments of this application.

For example, the target question is “What are the symptoms?”. The answer options may include: [symptom 1, symptom 2, . . . , symptom N, none of the foregoing symptoms]. Assuming that the user selects the answer option [symptom 1], “symptom 1” may be determined as the user disease feature. Assuming that the user selects the answer option [none of the foregoing symptoms], the inquiry of target question A misses the user disease feature.

In the embodiments of this application, disease features not present to the user may also be determined and recorded according to the selection operation of the user for the answer options, so as to carry the disease features not present to the user in an inquiry information processing result.

In the embodiments of this application, target disease features may be determined from the disease features corresponding to the candidate diseases, and target questions may be generated for the target disease features. The target disease features may be used for representing user disease features inquired in the current inquiry round.

According to an optional embodiment, the user disease features determined according to the user input may be removed from the disease features corresponding to the candidate diseases to obtain target disease features.

According to another optional embodiment, target disease features may be determined from the disease features corresponding to the candidate diseases according to importance scores corresponding to the disease features.

The factor features of the importance scores may specifically include at least one of the following features:
conditional probabilities of disease features under the condition of the diseases;
incidence probabilities of the diseases in a disease system;
a system probability of the disease system; and
a correlation between the disease features corresponding to the candidate diseases and the user disease features.

For example, target disease features may be determined from the disease features corresponding to the candidate diseases according to the correlation between the disease features corresponding to the candidate diseases and the user disease features determined according to the user input. For example, target disease features with the correlation greater than a first threshold may be determined from the disease features corresponding to the candidate diseases. In some embodiments, the correlation between the disease features may be determined according to co-occurrence information of multiple disease features in medical resources for a disease. It is to be understood that the embodiments of this application are not limited to the specific manner of determining the correlation between the disease features.

In an optional embodiment of this application, preset questions and target questions corresponding to a user may be saved to a question queue, an inquiry question may be taken from the question queue, and the inquiry question may be output to the user.

The questions in the question queue may have a priority, and the inquiry question may be taken from the question queue according to the priority of the questions. Determination factors for the priority of the questions may include: enqueue time of the questions, and/or matching degrees between disease features corresponding to the questions and the user disease features, etc. It is to be understood that the embodiments of this application are not limited to the specific process of taking an inquiry question from the question queue.

The execution body of the embodiments of this application may be a server or a client. When the execution body is the server, the server may be a processing engine or an interaction engine. The interaction engine may be configured to interact with the client, and the processing engine may transmit an inquiry question to the interaction engine, whereby the interaction engine provides the inquiry question to the user. It is to be understood that the embodiments of this application are not limited to the specific implementation of providing the inquiry question to the user.

In an optional embodiment of this application, after each execution of step 202, it may be judged whether to end the inquiry information processing according to information such as the scores of the candidate diseases, the user disease features, and the number of inquiry rounds. If the inquiry is ended, an inquiry information processing result is output, otherwise, step 203 is continued.

In the embodiments of this application, it is dynamically determined whether the inquiry stops in advance according to the predicted disease features, the user disease features, number of inquiry rounds, and other information, so as to improve the inquiry efficiency, thereby improving the user experience.

In summary, according to the inquiry information processing method in the embodiments of this application, disease prediction processing is dynamically performed based on user disease features obtained by at least one user input during an inquiry, and target questions are dynamically generated. Since the target questions for the inquiry are automatically generated during the inquiry in the embodiments of this application, the inquiry efficiency can be improved.

Furthermore, in the embodiments of this application, the process of performing disease prediction on the user disease features and generating target questions may be a dynamic process. Therefore, the target questions more related to the user disease features can be obtained according to the accumulation of the user disease features in the inquiry process, and the rationality of the inquiry according to the target questions can thus be improved. Moreover, candidate diseases more matching the user disease features can be obtained according to the accumulation of the user disease features in the inquiry process, and the accuracy of generating target questions for disease prediction can thus be improved.

### Method Embodiment 2

The present embodiment serves to illustrate a knowledge graph.

In the embodiments of this application, the knowledge graph is a structured semantic knowledge base for describing concepts and their interrelationships in the physical world.

In the embodiments of this application, an entity refers to something that exists objectively and may be distinguished from each other, including a specific person, thing, object, abstract concept or relationship, etc. The entity may be a specific object, such as: a disease or a disease feature, and may also be an abstract event, such as: an inquiry for disease features.

The entity may have many properties, a single property being referred to as an attribute. Each attribute has a range of values, the type of which may be integer, real number, string, etc. The named unit of a tag attribute is referred to as a field. The state of the field may include: a filled state or an unfilled state. The filled state corresponds to a filled field content, and the unfilled state represents that the corresponding field content is to be filled.

Entities in the medical field may be referred to as medical entities. The medical entities may include: disease entities, disease feature entities, or question entities, etc.

The disease entities may represent specific diseases, such as “hypertension” or “leukemia”. The diseases may correspond to a disease system. The disease system may correspond to an anatomical system. For example, the disease system may include: an exercise system, a digestive system, a respiratory system, a urinary system, a reproductive system, an endocrine system, an immune system, a nervous system, a circulatory system, etc.

In some embodiments, attributes of the disease entities may include at least one of the following attributes:
a disease identification attribute, a disease system attribute, a feature set attribute, a clinical proportion attribute, and a high incidence age attribute.

The feature set may include: disease features associated with the disease feature entities.

The clinical proportion attribute is used for representing incidence probabilities of diseases in the disease system, and may be obtained from the incidence rate of the diseases and the incidence rate of the disease system.

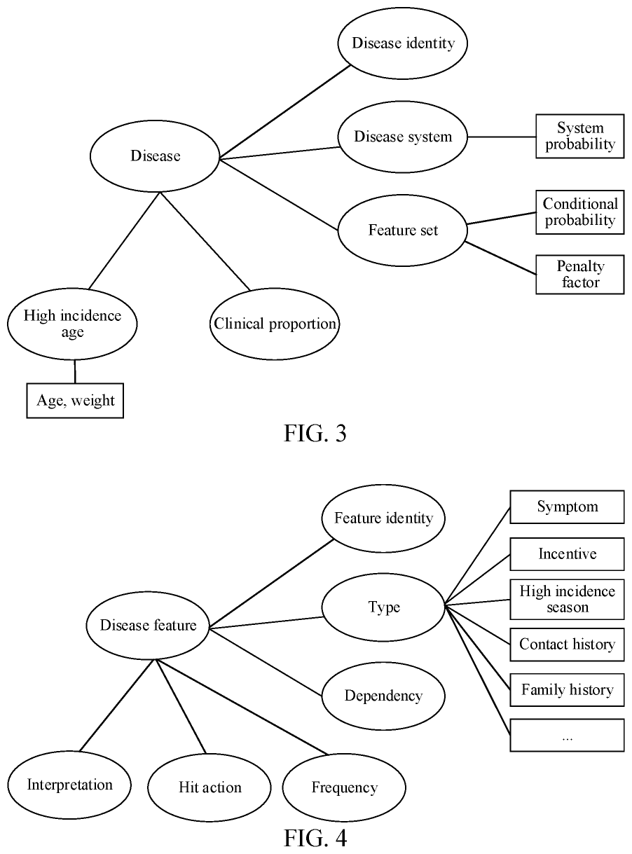

Referring to FIG. 3, a schematic diagram of a disease entity and attributes thereof according to an embodiment of this application is shown. The attributes of the disease entities may include: a disease identification attribute, a disease system attribute, a feature set attribute, a clinical proportion attribute, and a high incidence age attribute.

A single attribute may correspond to an attribute parameter.

For example, the attribute parameters of the attributes of the disease system include: a system probability, which may represent a proportion of patients of a single disease system to patients of all disease systems, and may be obtained according to a ratio of patients of a single disease system to patients of all disease systems.

For another example, the attribute parameters of the feature set attribute may include at least one of the following parameters:
conditional probabilities of disease features under the condition of the diseases, the feature set generally including multiple disease features, and the conditional probabilities being conditional probabilities respectively corresponding to the multiple disease features under the condition of the diseases; and
penalty factors of disease features under the condition of the diseases, the penalty factors corresponding to disease features not present under the condition of the diseases, and being used for penalizing the probabilities of the diseases during disease prediction.

Referring to Table 1, a schematic diagram of an instance of a disease entity according to an embodiment of this application is shown. Diseases with disease names “acute laryngitis” and “bronchitis” are both diseases of the “respiratory system” each corresponding to multiple disease features, each disease feature corresponding to a conditional probability.

TABLE 1

| | | | High | |
|---|---|---|---|---|
| | | | incidence | |
| Disease | Disease | Clinical | age | Feature set (disease features separated |
| name | system | proportion | weight | from conditional probabilities by #) |
| Acute | Respiratory | 0.04 | <5#0.8 | Pharyngalgia#0.8, hoarseness#0.8, |
| laryngitis | system | | | cough#0.6, barking cough*cough#0.8, |
| | | | | laryngeal stridor#0.7, dyspnea#0.2, |
| | | | | fever#0.6, incentive_catch cold or |
| | | | | cold#0.6, incentive_excessive sound#0.6, |
| | | | | high incidence season_spring#0.8, high |
| | | | | incidence season_winter#0.8, high |
| | | | | incidence season_summer#0.6, high |
| | | | | incidence season_autumn#0.6, onset |
| | | | | time_night#0.4, inspiratory effort or |
| | | | | inspiratory three depressions sign#0.3, |
| | | | | expectoration#0.4, congestion of |
| | | | | throat#0.8, phlegm stridor in |
| | | | | throat*laryngeal stridor#0.3, runny |
| | | | | nose#0.3, high fever*fever#0.5 |
| Bronchitis | Respiratory | 0.13 | <6#0.5 | Cough#0.8, expectoration#0.6, phlegm |
| | system | | | stridor in throat*laryngeal stridor#0.6, |
| | | | | laryngeal stridor#0.6, chest pain#0.3, |
| | | | | fever#0.6, vomiting#0.3, diarrhea#0.3, |
| | | | | medical history_upper respiratory tract |
| | | | | infection#0.8, incentive_weather |
| | | | | change#0.6, high incidence |
| | | | | season_winter#0.8, high incidence |
| | | | | season_spring#0.8, yellow |
| | | | | sputum*expectoration#0.5, runny |
| | | | | nose#0.5, antiadoncus#0.2 |

The disease feature entities may represent specific disease features. Types of the disease features may include: symptoms, incentives, high incidence seasons, contact history, family history, etc.

Attributes of disease feature entities may include: a hit action attribute for representing information of a triggered question entity when a corresponding disease feature entity is selected.

Attributes of disease feature entities may include: a dependency attribute for representing a disease feature entity having a parent-child relationship with a corresponding disease feature entity. Attributes of parameters of the dependency attribute may include: parent disease features or child disease features.

For example, the child disease feature of the disease feature “vomiting” includes: “projectile vomiting”. For another example, the child disease features of the disease feature “fever” include: “low fever”, “high fever”, etc.

Referring to FIG. 4, a schematic diagram of a disease feature entity and attributes thereof according to an embodiment of this application is shown. Attributes of disease feature entities may include: a feature identification attribute, a type attribute, a dependency attribute, a frequency attribute, a hit action attribute, an interpretation attribute, etc. The frequency attribute may represent the number of times the corresponding disease features are present in the feature set of all disease entities.

Referring to Table 2, a schematic diagram of an instance of a disease feature entity according to an embodiment of this application is shown. The named action attribute “question entity identity 18” of the disease feature “vomiting” represents that a question entity having a question entity identity of 18 will be triggered when the disease feature “vomiting” is selected.

TABLE 2

| Disease | | | | | |
|---|---|---|---|---|---|
| feature | Type | Dependency | Frequency | Hit action | Interpretation |
| Vomiting | Symptom | Projectile | 62 | Question | Vomiting |
| | | vomiting | | entity identity | |
| | | | | 18 | |
| Projectile | Symptom | Vomiting | 1 | None | Vomit farther directly |
| vomiting | | | | | without nausea |

The hit action attribute may improve the rationality of question succession in the inquiry procedure. For example, if the symptom “vomiting” is selected by the user, the corresponding question entity identity 18 is found according to the hit action attribute of the symptom “vomiting” for further inquiry of the symptom “projectile vomiting”.

The question entity corresponds to one inquiry of disease features and is used for representing questions corresponding to the inquiry. Since one inquiry may involve at least one disease feature, the question corresponding to the question entity may involve at least one disease feature.

In some embodiments, fields of the question entity may include: question text fields and answer option fields. The question text fields are used for representing the question to be answered. Examples of the question text fields may include: “Did you vomit directly without nausea?”, “What are the symptoms?”, “What diseases did you have before?”, “What is the type of rash?”, “What is the shape of stool”, etc. The answer option fields are used for representing available answer options.

In some embodiments, the fields of the question entity may further include at least one of the following fields: a disease feature field, a trigger condition field, and a jump relationship field.

The disease feature field is used for representing a disease feature entity.

The trigger condition field is used for representing that the corresponding question entity is obtained by triggering according to the disease feature entity.

The jump relationship field is used for executing a preset jump under the condition of selecting an answer option.

In some embodiments, the jump relationship field is used for jumping from a first question entity to a second question entity under the condition of selecting the answer option, and the disease feature entity corresponding to the first question entity and the disease feature entity corresponding to the second question entity are in a parent-child relationship.

Of course, those skilled in the art may determine a preset jump according to actual application requirements. For example, the preset jump may further include: executing a preset function which may be used for ending the search of a question entity, etc.

Referring to Table 3, the meanings and values of the fields of the question entity are shown.

TABLE 3

| Field | Meaning | Value |
|---|---|---|
| Identity | ID (Identity) | String |
| Question | Question text | String |
| text | | |
| Answer | Answer option | List of strings |
| option | | |
| Disease | Answer option or question | Identity of disease |
| feature | text | feature entity |
| | Corresponding disease | |
| | feature | |
| Trigger | If a disease feature that meets | Type of feature value; |
| condition | a trigger condition is selected, | or Parent-child relationship |
| | the question is automatically | (i.e. if a parent disease |
| | asked | feature is selected, a |
| | | question corresponding to a |
| | | child disease feature is |
| | | automatically asked) |
| Jump | If an answer option is | Identity of preset question |
| relationship | selected, a preset question | entity; preset function |
| | entity is executed; or, if | |
| | there is no preset question | |
| | entity, a preset function | |
| | (e.g. end function) is executed | |

The question entities in the embodiments of this application may include: a question entity instance, and/or a question entity template.

All fields of the question entity instances are in a filled state. The question entity instances may correspond to preset disease features.

Referring to Table 4, a schematic diagram of a question entity instance according to an embodiment of this application is shown. The identity of the question entity instance is 18, which is obtained by triggering according to the disease feature “vomiting”. That is, when the feature “vomiting” is selected, the question entity instance having an identity of 18 may be triggered.

The question entity instance corresponds to the disease feature “projectile vomiting” and is used for performing inquiry on the disease feature “projectile vomiting”. The interpretation of the disease feature “projectile vomiting” may be included in the question text, so as to assist the user in determining whether to hit the corresponding disease feature and selecting the corresponding answer option.

TABLE 4

| | |
|---|---|
| Identity | 18 |
| Question text | Did you vomit directly without nausea? |
| Answer option | [Yes, No, I don't know] |
| Disease feature | Projectile vomiting |
| Trigger condition | Vomiting |

Question text fields of the question entity templates are in a filled state, and preset fields other than the question text fields of the question entity templates are in an unfilled state. Preset fields may include: an answer option field, a disease feature field, a trigger condition field, a jump relationship field, etc.

The question entity templates may correspond to disease features of preset types. In this way, in the inquiry procedure, corresponding question entity templates may be found in the knowledge graph according to the types corresponding to user-related disease features, and field filling may be performed on the question entity templates according to the user-related disease features. The field-filled question entity templates may serve as dynamic question entity instances, and the dynamic question entity instances may contain questions for an inquiry. Since the question entity templates correspond to the disease features of preset types, the field-filled question entity templates may contain information of multiple disease features of preset types. Therefore, the number of disease features contained in the questions for an inquiry can be determined, thus reducing the number of interaction rounds of the inquiry and improving the inquiry efficiency.

The foregoing operation of performing field filling on the question entity templates may specifically include: filling an answer option field according to the user-related disease features. Different disease features may correspond to different answer options. Specifically, the answer option field may be filled with interpretations corresponding to the user-related disease features, and different interpretations may correspond to different answer options.

The foregoing operation of performing field filling on the question entity templates may specifically include: filling a jump relationship field according to a hit action attribute corresponding to the user-related disease features. Specifically, the jump relationship field may be filled with the hit action attribute corresponding to the user-related disease features.

For example, the content of the jump relationship field may be: when an answer option is selected, jumping to a preset question entity. Assuming that the answer option corresponds to a first disease feature entity and a second disease feature entity is recorded in a hit action attribute of the first disease feature entity, the preset question entity may be question entity information corresponding to the second disease feature entity.

Assuming that the user-related disease feature is a first disease feature, information of the first disease feature may be filled in the answer option field of the question entity template, and the question entity information corresponding to the second disease feature entity may be filled in the jump relationship field.

Referring to Table 5, a schematic diagram of a question entity template according to an embodiment of this application is shown. The question entity template may correspond to a disease feature of a symptom type for performing an inquiry on the disease feature of the symptom type.

In the inquiry procedure, field filling may be performed on the question entity template according to user-related symptom 1, symptom 2, . . . , symptom N (N may be a natural number greater than 0).

For example, interpretations of symptom 1, symptom 2, . . . , symptom N are filled in the answer option field, so as to assist the user in determining whether to hit the corresponding disease feature and selecting the corresponding answer option.

It is to be noted that in the embodiments of this application, when the answer options correspond to the disease features on a one-to-one basis, “selecting answer options” and “selecting disease features” may be equivalent features. For example, selecting an answer option corresponding to symptom 1 may be equivalent to selecting symptom 1.

For another example, symptom types may be filled in the disease feature field. Or, question entity identities corresponding to child symptoms of symptom 1, symptom 2, . . . , symptom N, etc. are filled in the jump relationship field.

It is to be understood that the filling of other preset fields than the filling of the answer option field is optional. That is, the filling of the disease feature field, the trigger condition field and the jump relationship field may not be performed.

It is to be understood that the question entity template corresponding to the disease feature of the symptom type shown in Table 5 is merely provided as an optional embodiment, and in fact, those skilled in the art may also adopt question entity templates corresponding to disease features of other types according to practical application requirements. For example, a question entity template corresponding to a disease feature of a contact history type may also be adopted, and a corresponding question text may include: “Have you been in contact with patients having the following pathogens, harmful factors and diseases?” etc.

TABLE 5

| | |
|---|---|
| Identity | ID |
| Question text | What are the symptoms? |
| Answer option | Interpretations of symptom 1, symptom |
| | 2, . . . , symptom N |
| Disease feature | Symptom type |
| Jump relationship | When an answer option is selected, jump |
| | to a preset question entity, or end the |
| | search for a question entity |

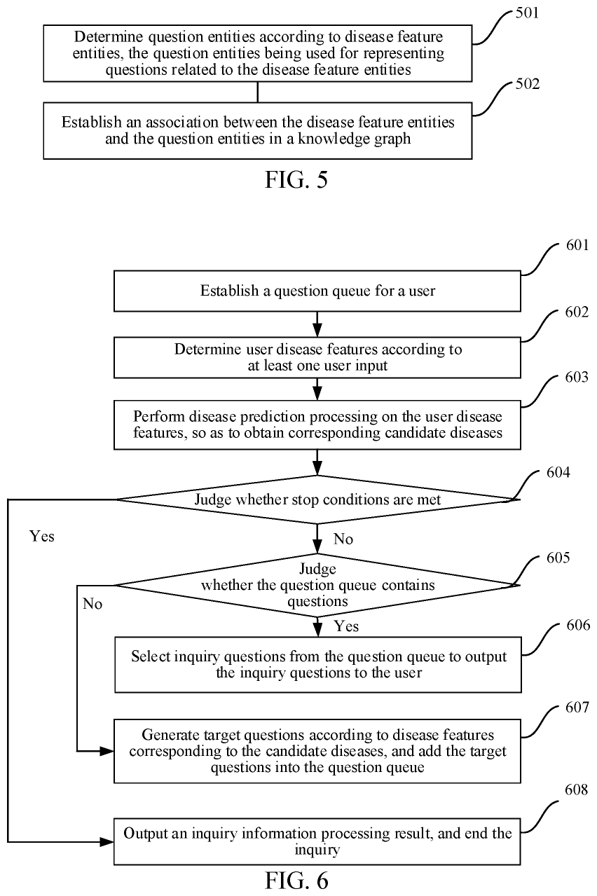

Referring to FIG. 5, a flowchart of steps of a knowledge graph processing method according to an embodiment of this application is shown. The method may specifically include the following steps:

Step 501: Determine question entities according to disease feature entities, the question entities being used for representing questions related to the disease feature entities.

Step 502: Establish an association between the disease feature entities and the question entities in a knowledge graph.

In step 501, the disease feature entities may represent disease-related features, which may include: disease features present in the diseases and may further include: disease features not present in the diseases.

In an optional embodiment of this application, the process of determining disease feature entities may include: determining a chief complaint list, and determining a disease list corresponding to the chief complaint list; expanding disease features of the diseases in the disease list according to medical resources; and determining disease feature entities according to chief complaints in the chief complaint list and the filled disease features.

In the field of medical technology, the chief complaint is used for representing narration of a patient or a surrogate for the most prominent symptoms and/or signs, and the chief complaint typically includes: at least one of the symptoms and signs stated by the patient or the surrogate, nature, and duration.

In the embodiments of this application, a chief complaint may be acquired from medical resources such as medical query data and/or medical record data, and a chief complaint list may be established according to the obtained chief complaint.

In a specific implementation, a corresponding disease may be determined according to a single chief complaint in the chief complaint list, and the determined disease may be added to the disease list. One implementation may be: transmitting a chief complaint to a doctor terminal, and determining a disease corresponding to the chief complaint by a user corresponding to the doctor terminal. The user of the doctor terminal may be a doctor with clinical experience of more than M (M may be a natural number greater than 0, e.g. M may be greater than 7) years, and may determine the disease corresponding to the chief complaint based on knowledge and experience.

The diseases in the disease list may serve as data sources for disease entities in the knowledge graph. That is, corresponding disease entities may be constructed according to the diseases in the disease list.

After determining the disease list, disease features may be expanded for diseases in the disease list according to medical resources such as medical books, medical databases and medical question and answer data in the embodiments of this application. That is, for a disease, disease features other than a chief complaint corresponding to the disease are expanded on the basis of the chief complaint. Types of the disease features involved in the expansion may include: symptoms, incentives, high incidence seasons, contact history, family history, etc.

In some embodiments, disease contents corresponding to diseases may be obtained from medical resources, and disease features corresponding to the foregoing types may be extracted from the disease contents.

The chief complaints in the chief complaint list and the filled disease features may serve as data sources of the corresponding disease features of the disease feature entities. That is, the disease feature entities may also be constructed according to the chief complaints in the chief complaint list and the filled disease features.

In an optional embodiment of this application, candidate disease features corresponding to the diseases (the chief complaints and the filled disease features) may also be transmitted to the doctor terminal whereby the user of the doctor terminal updates the candidate disease features. The updating of the candidate disease features may specifically include: addition of the candidate disease features, deletion of the candidate disease features, or modification of the candidate disease features, etc.

The updated candidate disease features may serve as data sources of the corresponding disease features of the disease feature entities. For example, a feature set corresponding to the diseases may be determined for the updated candidate disease features corresponding to the diseases.

In an optional embodiment of this application, conditional probabilities of the disease features in the feature set under the condition of the diseases and/or penalty factors of the disease features under the condition of the diseases may also be determined for the diseases. In some embodiments, the conditional probabilities or penalty factors may be determined according to the occurrence information of disease features in medical resources corresponding to the diseases; or, the conditional probabilities or penalty factors may be determined by the user of the doctor terminal.

In yet another optional embodiment of this application, incidence probabilities (clinical proportion) of the diseases in the disease system and/or a system probability may also be determined according to the diseases and the medical resources of the disease system to which the diseases belong.

The conditional probabilities may represent matching degrees between disease features and diseases or importance degrees of disease features to diseases. Therefore, applying the conditional probabilities to disease prediction processing can improve the discrimination of multiple candidate diseases when the user disease features correspond to multiple candidate diseases.

In application example 1 of the embodiments of this application, in the process of disease prediction for a patient who has the symptoms “cough” and “expectoration”, although “acute laryngitis” and “bronchitis” both match the two symptoms, the probability of “cough” in “acute laryngitis” is 0.6, and the probability of “expectoration” in “acute laryngitis” is 0.4. However, the probability of “cough” in “bronchitis” is 0.8, and the probability of “expectoration” in “bronchitis” is 0.6. Due to the conditional probabilities, it may be determined that the matching degrees between the two symptoms and “bronchitis” are higher than the matching degrees between the two symptoms and “acute laryngitis”, thereby improving the discrimination between multiple candidate diseases when the user disease features correspond to multiple candidate diseases.

The penalty factors can represent the extent to which disease features are excluded from diseases, and thus can comprehensively determine the influence of multiple disease features on the diseases. For example, if a user has a feature not to be present in a candidate disease, the probability of the candidate disease may be reduced according to a penalty factor. For example, the score of the candidate disease may be reduced according to the penalty factor, and the accuracy of the probability of the candidate disease under the condition of user disease features can be improved. Thus, the discrimination between multiple candidate diseases can be improved when the user disease features correspond to the multiple candidate diseases.

The clinical proportion can represent incidence probabilities of diseases in the disease system, and may reflect the commonness of the corresponding diseases. Applying the clinical proportion to the disease prediction processing can improve the accuracy of the probabilities of candidate diseases under the condition of the user disease features. For example, when the user disease features correspond to multiple candidate diseases, the multiple candidate diseases may be ranked according to corresponding clinical proportions of the multiple candidate diseases. Thus, the discrimination between the multiple candidate diseases can be improved when the user disease features correspond to the multiple candidate diseases.

In the process of applying the clinical proportion and the system probability to disease prediction, prior probabilities of the candidate diseases may be determined according to the clinical proportion and the system probability, and then multiple candidate diseases may be ranked according to the prior probabilities. In this way, the accuracy of the probabilities of the candidate diseases under the condition of user disease features can be improved. Thus, the discrimination between multiple candidate diseases can be improved when the user disease features correspond to the multiple candidate diseases.

In an optional embodiment of this application, the operation of determining disease feature entities may specifically include: performing feature normalization on the chief complaints and the filled disease features so as to obtain normalized disease features; and determining disease feature entities according to the normalized disease features.

The feature normalization may unify disease features with the same semantics and different descriptions into a standard description. For example, personalized or colloquial symptom descriptions corresponding to “headache” specifically include: “pricking pain”, “throbbing pain”, “touching pain”, “swallowing pain”, etc. For another example, personalized or colloquial symptom descriptions corresponding to “tongue pain” specifically include: “pain on the left side of the tongue”, “pain on the tip of the tongue”, “pain on the root of the tongue”, “pain on the edge of the tongue”, etc.

In the embodiments of this application, the operation of determining disease feature entities may specifically include: determining multiple attributes corresponding to disease feature entities, and determining corresponding attribute values for a particular disease feature entity. The multiple attributes corresponding to disease feature entities may specifically include: a feature identification attribute, a type attribute, a dependency attribute, a frequency attribute, a hit action attribute, an interpretation attribute, etc.

The question entities in the embodiments of this application are used for performing an inquiry on disease symptoms corresponding to the disease feature entities, so as to assist the user in determining whether corresponding disease symptoms are present.

Those skilled in the art may determine question entities corresponding to the disease feature entities according to practical application requirements. According to one embodiment, information of the disease feature entities may be transmitted to the doctor terminal whereby the doctor sets question entities corresponding to the disease feature entities.

According to another embodiment, question entities corresponding to the disease feature entities may be determined according to the types corresponding to the disease feature entities and historical inquiry data.

In the embodiments of this application, fields of the question entities may include: a question text field and an answer option field. The question text field or the answer option field may include information such as identities or interpretations of the disease feature entities.

In some embodiments, the fields of the question entity may further include at least one of the following fields: a disease feature field, a trigger condition field, and a jump relationship field.

The disease feature field is used for representing a disease feature entity.

The trigger condition field is used for representing that the corresponding question entity is obtained by triggering according to the disease feature entity.

The jump relationship field is used for executing a preset jump under the condition of selecting an answer option.

In some embodiments, the jump relationship field is used for jumping from a first question entity to a second question entity under the condition of selecting the answer option, and the disease feature entity corresponding to the first question entity and the disease feature entity corresponding to the second question entity are in a parent-child relationship.

Of course, those skilled in the art may determine a preset jump according to actual application requirements. For example, the preset jump may further include: executing a preset function which may be used for ending the search of a question entity, etc.

In the embodiments of this application, information of the question entities may also be transmitted to the doctor terminal whereby the user of the doctor terminal audits the information of the question entities.

In step 502, the question entities may include: question entity instances. The question entity instances may correspond to preset disease features. Therefore, in the embodiments of this application, an association between preset disease feature entities and the question entity instances may be established in the knowledge graph.

The question entities in the embodiments of this application may include: question entity templates. The question entity templates may correspond to disease features of preset types. Therefore, in the embodiments of this application, an association between disease feature entities of preset types and the question entity instances may be established in the knowledge graph.

In the embodiments of this application, the operation of establishing an association between disease feature entities and question entities may specifically include: establishing a mapping relationship between the disease feature entities and the question entities according to the disease feature fields in the question entities. The disease feature fields in the question entities match the disease feature entities.

If the disease feature fields in the question entities represent preset disease features, the question entities in the mapping relationship are question entity instances, and the disease feature entities in the mapping relationship correspond to a preset disease feature.

If the disease feature fields in the question entities represent disease features of preset types, the question entities in the mapping relationship are question entity templates, and the disease feature entities in the mapping relationship correspond to multiple disease features of preset types.

In an optional embodiment of this application, an association between disease entities and disease feature entities may also be established in the knowledge graph. Specifically, the disease entities may be associated with disease feature entities corresponding to disease features in a feature set thereof. In this way, the user disease features may match the feature set corresponding to the disease. It is to be understood that the embodiments of this application are not limited to the specific manner of associating the disease entities and the disease feature entities.

In summary, according to the knowledge graph processing method in the embodiments of this application, an association between the disease feature entities and the question entities may be established in the knowledge graph. In this way, in the inquiry procedure, corresponding question entities may be found in the knowledge graph according to user-related disease features, and questions for the inquiry may be obtained according to the found question entities. Since the knowledge graph in the embodiment of this application includes an association between the disease feature entities and the question entities and questions for the inquiry may be generated in the inquiry procedure, the effect of the knowledge graph on the inquiry can be enhanced, thereby improving the efficiency and accuracy of inquiry information processing according to the knowledge graph.

Furthermore, in the embodiments of this application, the hit action attribute in the disease feature entities represents a relationship between the disease features and the information of the question entities, and the corresponding question entities will be triggered when the corresponding disease features are selected. Since the association between the disease features and the question entities may be automated, the cost of resources for manual operation of an inquiry path can be reduced, and the rationality of question succession in the inquiry procedure can also be improved.

In addition, in the embodiments of this application, the jump relationship field in the question entities represents jump to preset question entities when answer options corresponding to the disease features are selected. In this way, the rationality of question succession in the inquiry procedure can be improved. Since the association between the disease features and the question entities may be automated, the cost of resources for manual operation of an inquiry path can be reduced, and the rationality of question succession in the inquiry procedure can also be improved.

Furthermore, in the embodiments of this application, attribute parameters such as conditional probabilities, penalty factors, clinical proportions, and system probabilities are set in the disease entities. The conditional probabilities can represent matching degrees between disease features and diseases or importance degrees of disease features to diseases. The penalty factors can represent the exclusion degree of disease features to diseases, and then can comprehensively determine the influence of multiple disease features to diseases. The clinical proportions can represent the incidence probabilities of diseases in the disease system and may reflect the commonness of the corresponding diseases. Applying one or more of the attribute parameters to disease prediction processing may reflect the probabilities of corresponding candidate diseases, thereby improving the accuracy of the probabilities of the candidate diseases under the condition of the user disease features.

### Method Embodiment 3

In the present embodiment, disease prediction processing in the process of inquiry information processing is described.

In the embodiments of this application, an association relationship between disease entities and disease feature entities included in a knowledge graph may be searched for candidate diseases according to user disease features obtained by a user input. Specifically, the user disease features may match the disease feature entities in the association relationship, and diseases corresponding to the disease entities matching successfully may serve as candidate diseases.

In the embodiments of this application, the user disease features may match at least one candidate disease. In the embodiments of this application, probabilities of the candidate diseases may be represented according to scores of the candidate diseases, and the candidate diseases may be screened according to the scores of the candidate diseases.

In an optional embodiment of this application, the scores of the candidate diseases may be determined according to probability features.

The probability features may be features recorded in the knowledge graph, and may specifically include at least one of the following features:
conditional probabilities of the disease features matching the user disease features under the condition of the candidate diseases;
penalty factors of the disease features matching the user disease features under the condition of the candidate diseases;
incidence probabilities of the candidate diseases in a disease system;
and an incidence probability of the disease system.

According to one embodiment, the scores of the candidate diseases may be determined according to the conditional probabilities.

In application example 1 of the embodiments of this application, the user disease features include two symptoms “cough” and “expectoration”. In the process of disease prediction, although “acute laryngitis” and “bronchitis” both match the two symptoms, the probability of “cough” in “acute laryngitis” is 0.6, and the probability of “expectoration” in “acute laryngitis” is 0.4. However, the probability of “cough” in “bronchitis” is 0.8, and the probability of “expectoration” in “bronchitis” is 0.6. According to the conditional probabilities, it may be determined that the matching degrees between the two symptoms and “bronchitis” are higher than the matching degrees between the two symptoms and “acute laryngitis”. Therefore, it may be determined that the score of “bronchitis” is higher than the score of “acute laryngitis”.

The penalty factors may correspond to disease features not present under the condition of the candidate diseases, and are used for penalizing the probabilities of the candidate diseases during disease prediction. For example, the candidate diseases include: candidate disease A, but the user disease features include: disease feature X not to be present in candidate disease A. In this case, a score of candidate disease A can be reduced according to corresponding penalty factors of disease feature X and candidate disease A.

The clinical proportion can represent incidence probabilities of diseases in the disease system, and may reflect the commonness of the corresponding diseases. Applying the clinical proportion to the disease prediction processing can improve the accuracy of the score of the disease prediction. Usually, as the clinical proportion is higher, the score of the corresponding candidate disease is higher.

In the process of applying the clinical proportion and the system probability to disease prediction, prior probabilities of the candidate diseases may be determined according to the clinical proportion and the system probability, and then the scores of the candidate diseases may be determined according to the prior probabilities. Usually, as the prior probabilities of the candidate diseases are higher, the scores of the candidate diseases are higher.

When multiple probability features are adopted, the multiple probability features may be combined, and the scores of the candidate diseases may be determined according to the combined probability features. Corresponding combination modes may include: a weighted average mode, or a product mode, etc.

In the embodiments of this application, the operation of screening candidate diseases according to the scores of the candidate diseases may specifically include: selecting candidate diseases having scores greater than a score threshold, and/or, selecting candidate diseases having scores ranked in the first P (P may be a natural number greater than 0).

Assuming that the screened candidate diseases are target candidate diseases, target questions may be generated according to disease features corresponding to the target candidate diseases, or information of the target candidate diseases may be output as an inquiry information processing result.

### Method Embodiment 4

In the present embodiment, stop conditions for inquiry information processing are described.

The stop conditions may represent conditions corresponding to stop of the inquiry information processing. When the inquiry information processing is stopped, the output of target questions to a user may be stopped, and a corresponding inquiry information processing result may also be output to the user.

Correspondingly, the foregoing method may further include: stopping executing the operation of generating target questions in response to meeting stop conditions upon obtaining candidate diseases.

Correspondingly, the foregoing method may further include: outputting an inquiry information processing result in response to meeting the stop conditions upon obtaining the candidate diseases. The inquiry information processing result may include: information of the candidate diseases. Information of one or more target candidate diseases, such as the name and score of the target candidate disease with the highest score, may be carried in the inquiry information processing result.

In some embodiments, the inquiry information processing result may further include: disease features present to the user and disease features not present to the user. Conditional probabilities of disease features present to the user under the condition of the target candidate diseases may also be carried in the inquiry information processing result.

It is to be understood that those skilled in the art may carry the required information in the inquiry information processing result according to practical application requirements, and the embodiments of this application are not limited to the specific information carried in the inquiry information processing result.

In the embodiments of this application, the stop conditions may optionally include at least one of the following conditions:
a score of at least one candidate disease is greater than a score threshold, which may be used for improving the quality of the target candidate diseases carried in the inquiry information processing result; and
the difference between scores of multiple candidate diseases meets difference conditions that may represent that the scores of at least two candidate diseases are different. When the difference conditions are not met, it is indicated that the user disease features correspond to multiple candidate diseases with similar scores. In this case, it is necessary to further screen the multiple candidate diseases with similar scores according to the user disease features.

An inquiry proportion of the disease features corresponding to the candidate diseases meets a proportion condition. The inquiry proportion may represent a proportion of the inquired disease features to all the disease features, and the proportion condition may be that the inquiry proportion is greater than a proportion threshold, etc.

The number of inquiry rounds exceeds a round threshold. When the number of inquiry rounds exceeds the round threshold, the inquiry is stopped, thereby saving the inquiry time and improving the user experience.

It is to be noted that when the stop conditions are not met, inquiry questions may be selected from the question queue, and the selected inquiry questions may be output to the user. If the question queue does not contain questions, target questions may be generated according to the disease features corresponding to the candidate diseases, and the generated target questions may be saved to the question queue.

Referring to FIG. 6, a flowchart of steps in Embodiment 4 of an inquiry information processing method according to this application is shown. The method may specifically include the following steps:

Step 601: Establish a question queue for a user. The question queue may include preset questions.

Step 602: Determine user disease features according to at least one user input.

Step 603: Perform disease prediction on the user disease features to obtain corresponding candidate diseases.

Step 604: Judge whether stop conditions are met, if yes, perform step 608, otherwise, perform step 605.

Step 605: Judge whether the question queue contains questions, if yes, perform step 606, otherwise, perform step 607.

Step 606: Select inquiry questions from the question queue to output the inquiry questions to the user.

Step 607: Generate target questions according to disease features corresponding to the candidate diseases, and add the target questions into the question queue.

Step 608: Output an inquiry information processing result, and end the inquiry.

### Method Embodiment 5

In the present embodiment, the process of generating target questions is described.

In the embodiments of this application, an association between disease feature entities and question entities is established in a knowledge graph. In this way, in the inquiry procedure, corresponding question entities may be found in the knowledge graph according to disease features corresponding to candidate diseases, and target questions for the inquiry may be obtained according to the found question entities. Since the knowledge graph in the embodiment of this application includes an association between the disease feature entities and the question entities and questions for the inquiry may be automatically generated in the inquiry procedure, the effect of the knowledge graph on the inquiry can be enhanced, thereby improving the inquiry efficiency and the accuracy of target questions for the inquiry.

In the embodiments of this application, a mapping relationship between disease feature entities and question entities included in the knowledge graph may be searched for question entities corresponding to the disease features according to the disease feature entities corresponding to the disease features.

The embodiments of this application may provide the following technical solutions for generating target questions:

Technical Solution 1

In technical solution 1, question entity instances corresponding to disease features may be determined according to a mapping relationship between disease feature entities and question entity instances.

In a specific implementation, the content of a question text field and the content of an answer option field may be acquired from the question entity instances corresponding to target disease features so as to obtain target questions. That is, the target questions may include: the content of the question text field and the content of the answer option field. For example, when the target disease feature is “vomiting”, target question A may be determined according to the question entity instance corresponding to “vomiting”. The question text of target question A may be “Did you vomit”, and the answer option of target question A may be: [Yes, No, I don't know].

Corresponding question entities may be acquired from the knowledge graph according to hit action attributes in the disease feature entities corresponding to the target disease features when answer options corresponding to the target disease features are selected.

For example, when the target disease feature is “vomiting” and the answer option [Yes] corresponding to target question A is selected, a question entity instance having a question entity identity of 18 will be triggered according to the hit action attributes of the disease feature entities shown in Table 2. In this case, target question B may be generated according to the question entity instance having a question entity identity of 18. The question text of target question B may be: “Did you vomit directly without nausea?” The answer option of target question B may be: [Yes, No, I don't know].

Technical Solution 2

In technical solution 2, question entity templates corresponding to disease features may be determined according to a mapping relationship between types of disease feature entities and question entity templates.

In this case, the operation of generating target questions may specifically include: performing field filling on the question entity templates according to the disease features to obtain target questions.

Fields of the question entity templates specifically include: question text fields and answer option fields. The operation of performing field filling on the question entity templates specifically includes: step S1 and/or step S2. A sequence of steps S1 and S2 may not be fixed.

Step S1 may be: performing, according to at least one disease feature of a type, field filling on the question entity template corresponding to the type, so as to carry information of the at least one disease feature in the obtained target questions. For example, if the type is “symptom”, interpretations of symptom 1, symptom 2, . . . , symptom N may be filled in the question entity templates corresponding to “symptom”, so as to assist a user in determining whether to hit the corresponding disease feature and selecting the corresponding answer option.

Step S2 may be: filling jump relationship fields of the question entity templates according to hit action attributes in the disease feature entities corresponding to the disease features. Specifically, the jump relationship field may be filled with information of the hit action attribute corresponding to the disease features.

For example, the content of the jump relationship field may be: when an answer option is selected, jumping to a preset question entity. Assuming that the answer option corresponds to a first disease feature entity and a second disease feature entity is recorded in a hit action attribute of the first disease feature entity, the preset question entity may be question entity information corresponding to the second disease feature entity.

Assuming that the disease feature is a first disease feature, information of the first disease feature may be filled in the answer option field of the question entity template, and the question entity information corresponding to the second disease feature entity may be filled in the jump relationship field.

Technical Solution 3

In technical solution 3, corresponding question entities may be acquired from a knowledge graph according to jump relationship fields in question entities corresponding to the disease features when answer options corresponding to the disease features are selected.

The jump relationship field is used for executing a preset jump under the condition of selecting an answer option. In some embodiments, the jump relationship field is used for jumping from a first question entity to a second question entity under the condition of selecting the answer option, and the disease feature entity corresponding to the first question entity and the disease feature entity corresponding to the second question entity may be in a parent-child relationship.

For example, when the answer option [Yes] corresponding to target question B is selected, a question entity instance corresponding to a symptom such as “lethargy” may be triggered according to the jump relationship field of the question entity. In this case, target question C may be generated according to the question entity instance corresponding to the symptom such as “lethargy”. The question text of target question C may be: “Are you lethargic?” The answer option of target question C may be: [Yes, No, I don't know].

Technical solution 1 determines a target question based on a question entity instance. Technical solution 2 determines a target question based on the filling of a question entity template. Technical solution 3 may acquire a question entity through a jump relationship field in the question entity. It is to be understood that those skilled in the art may use any one or a combination of technical solutions 1 to 3 according to practical application requirements.

In summary, according to the inquiry information processing method in the embodiments of this application, an association between the disease feature entities and the question entities may be established in the knowledge graph. In this way, in the inquiry procedure, corresponding question entities may be found in the knowledge graph according to disease features corresponding to candidate diseases, and target questions for the inquiry may be obtained according to the found question entities. Since the knowledge graph in the embodiment of this application includes an association between the disease feature entities and the question entities and target questions for the inquiry may be generated in the inquiry procedure, the effect of the knowledge graph on the inquiry can be enhanced, thereby improving the inquiry efficiency and the rationality of target questions.

Furthermore, in the embodiments of this application, the hit action attribute in the disease feature entities represents a relationship between the disease features and the information of the question entities, and the corresponding question entities will be triggered when the corresponding disease features are selected. Since the association between the disease features and the question entities may be automated, the cost of resources for manual operation of an inquiry path can be reduced, and the rationality of question succession in the inquiry procedure can also be improved.

In addition, in the embodiments of this application, the jump relationship field in the question entities represents jump to preset question entities when answer options corresponding to the disease features are selected. In this way, the rationality of question succession in the inquiry procedure can be improved. Since the association between the disease features and the question entities may be automated, the cost of resources for manual operation of an inquiry path can be reduced, and the rationality of question succession in the inquiry procedure can also be improved.

### Method Embodiment 6

Referring to FIG. 7, a flowchart of steps in Embodiment 6 of an inquiry information processing method according to this application is shown. The method may specifically include the following steps:

Step 701: Acquire question entities corresponding to preset disease feature entities from a knowledge graph according to user disease features obtained by at least one user input, the question entities being used for representing questions related to the preset disease feature entities.

Specifically, preset disease feature entities matching the user disease features are acquired from the knowledge graph, and then question entities corresponding to the matched preset disease feature entities are acquired.

Step 702: Determine preset disease questions according to the question entities, the preset disease questions specifically including: question text and at least one preset option.

Step 703: Output corresponding medical advice information in response to receiving a selection operation of a user for any preset option.

In the embodiments of this application, the preset disease feature entities may represent disease features corresponding to preset diseases. The preset diseases may include: diseases with higher emergency and/or severity. The disease features corresponding to the preset diseases may include: emergency and severe cases.

In the embodiments of this application, preset disease questions are determined according to the question entities corresponding to the preset disease feature entities during the inquiry, and a user is inquired using the preset disease questions. The preset diseases with higher emergency and/or severity may be excluded during the inquiry, which can improve the safety of the inquiry information processing.

According to one embodiment, a corresponding relationship between disease features and question entities corresponding to preset disease feature entities may be established. In this way, user disease features may match the disease features in the corresponding relationship to obtain question entities corresponding to the user disease features and the preset disease feature entities.

According to another embodiment, hit action attributes in the disease feature entities represent a relationship between disease features and information of question entities corresponding to preset disease feature entities. In this way, hit action attributes in the corresponding disease feature entities may be found according to the user disease features, and then question entities corresponding to the user disease features and the preset disease feature entities may be obtained.

In application example 2 of this application, assuming that the user disease feature is “fever”, a question entity corresponding to a preset disease related to “fever” may be determined according to the foregoing corresponding relationship or a hit action attribute corresponding to the entity “fever” in the embodiments of this application. For example, the question entity may include preset disease questions, and the question text of the preset disease questions may be “What are the symptoms?”. At least one preset option of the preset disease questions may include: [options corresponding to the preset disease features, none]. The options corresponding to the preset disease features may include: [over 40° C., chills], etc.

If a selection operation of a user for any preset option is received, it indicates that the user has hit the preset disease features, and indicates that the user has a high probability of corresponding preset diseases. In this case, the output of medical advice information can improve the degree of attention of the user to the disease condition, and thus can enhance the security of the information processing inquiry.

In summary, according to the knowledge graph processing method in the embodiments of this application, preset disease questions are determined according to the question entities corresponding to the preset disease feature entities during the inquiry, and a user is inquired using the preset disease questions. The preset diseases with higher emergency and/or severity may be excluded during the inquiry, which can improve the safety of the inquiry information processing.

### Method Embodiment 7

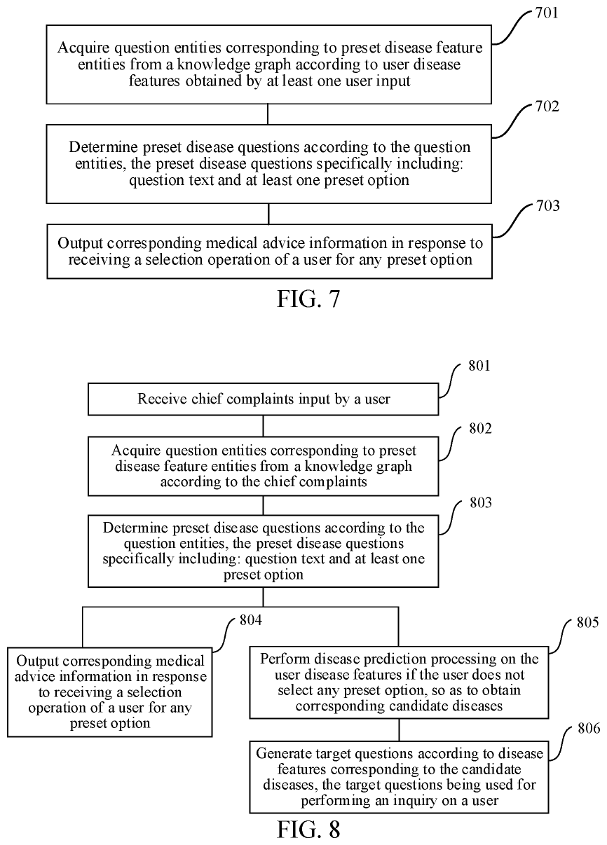

Referring to FIG. 8, a flowchart of steps in Embodiment 7 of an inquiry information processing method according to this application is shown. The method may specifically include the following steps:

Step 801: Receive chief complaints input by a user.

Step 802: Acquire question entities corresponding to preset disease feature entities from a knowledge graph according to the chief complaints, the question entities being used for representing questions related to the preset disease feature entities.

Step 803: Determine preset disease questions according to the question entities, the preset disease questions specifically including: question text and at least one preset option.

Step 804: Output corresponding medical advice information in response to receiving a selection operation of a user for any preset option.

Step 805: Perform disease prediction on the user disease features if the user does not select any preset option to obtain corresponding candidate diseases.

Step 806: Generate target questions according to disease features corresponding to the candidate diseases, the target questions being used for performing an inquiry on a user.

In the inquiry procedure, in the embodiments of this application, before a user is inquired, preset diseases may be excluded according to chief complaints, and if a selection operation of the user for any preset option is received, it indicates that the user has a high probability of having the preset diseases. In this case, corresponding medical advice information may be output, so as to improve the safety of the inquiry information processing.

If the user does not select any preset option, it indicates that the probability that the user has preset diseases is low, and the user may be inquired.

### Method Embodiment 8

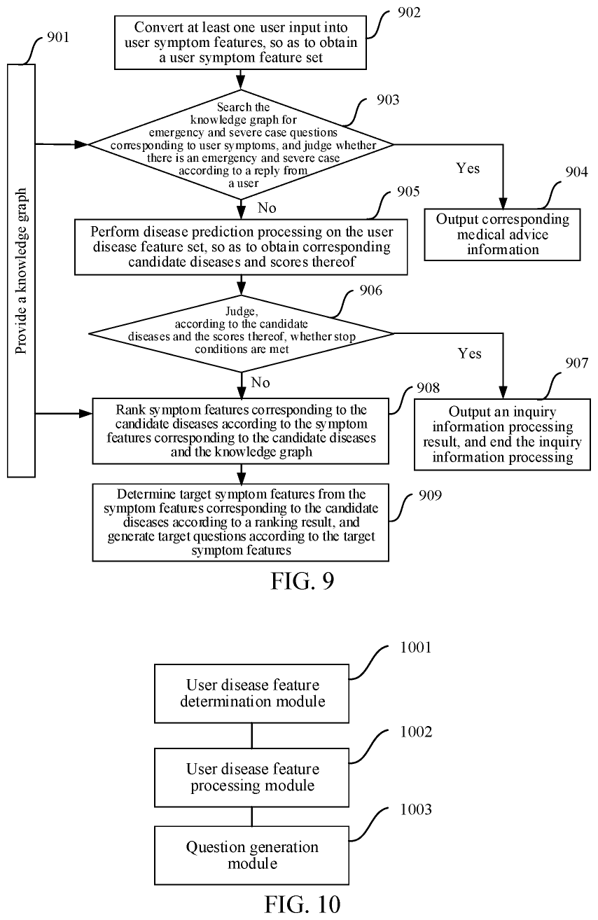

Referring to FIG. 9, a flowchart of steps in Embodiment 8 of an inquiry information processing method according to this application is shown. The method may specifically include the following steps:

Step 901: Provide a knowledge graph.

Step 902: Convert at least one user input into user symptom features to obtain a user symptom feature set.

Step 903: Search the knowledge graph for emergency and severe case questions corresponding to user symptoms, judge whether there is an emergency and severe case according to a reply from a user, if yes, perform step 904, otherwise, perform step 905.

Step 904: Output corresponding medical advice information.

Step 905: Perform disease prediction on the user disease feature set to obtain corresponding candidate diseases and scores thereof.

Step 906: Judge, according to the candidate diseases and the scores thereof, whether stop conditions are met, if yes, perform step 907, otherwise, perform step 908.

Step 907: Output an inquiry information processing result, and end the inquiry information processing.

Step 908: Rank symptom features corresponding to the candidate diseases according to the symptom features corresponding to the candidate diseases and the knowledge graph.

Step 909: Determine target symptom features from the symptom features corresponding to the candidate diseases according to a ranking result, and generate target questions according to the target symptom features, the target questions being used for collecting information such as the user symptoms.

In step 902, at least one user input may be converted into corresponding user symptom features using a symptom identification method such as an entity identification method, or a matching method of a disease feature table.

In practical applications, the operation of obtaining emergency and severe case questions corresponding to user symptoms in step 903 may specifically include: acquiring question entities corresponding to the corresponding emergency and severe case questions from the knowledge graph according to the user symptom feature set, the question entities representing various types of information contained in a question, including question text, an answer option and a jump relationship (i.e. a corresponding action when the answer option is selected, etc.).

The emergency and severe case questions are determined according to the question entities. Answer options of the emergency and severe case questions may include: at least one emergency and severe case option.

If a selection operation of the user for any emergency and severe case option is received, it may be indicated that the user symptom feature set corresponds to emergency and severe cases.

In practical applications, the process of performing disease prediction in step 905 specifically includes: determining candidate diseases corresponding to the current user symptom feature set according to matching information between the user symptom feature set and features such as symptoms corresponding to the diseases in a medical knowledge graph.

In practical applications, the process of performing disease prediction in step 905 specifically includes: determining scores of the candidate diseases according to probability features.

The probability features may specifically include at least one of the following features:
conditional probabilities of the disease features matching the user symptom feature set under the condition of the candidate diseases;
penalty factors of the disease features matching the user symptom feature set under the condition of the candidate diseases;
a clinical proportion of candidate diseases to all diseases of an anatomic system;
and a clinical proportion of corresponding diseases of various anatomic systems.

In step 906, the stop conditions may include at least one of the following conditions:
a score of at least one candidate disease is greater than a score threshold;
the difference between scores of multiple candidate diseases meets difference conditions;
an inquiry proportion of the symptom features corresponding to the candidate diseases meets a proportion condition; and
the number of inquiry rounds exceeds a round threshold.

In practical applications, the process of ranking the symptom features corresponding to the candidate diseases in step 908 specifically includes: calculating an importance score of each symptom feature according to the conditional probabilities of the disease feature entities corresponding to the symptom features contained in the candidate diseases to various diseases and information such as prior probabilities of the candidate diseases, and ranking the symptom features contained in the candidate diseases according to the importance score. In some embodiments, a target symptom feature meeting the criteria may be selected for the inquiry according to a preset criteria based on the ranking result. For example, the preset criteria may include: the importance score is greater than a second threshold, or the features are ranked in the first X (X may be a natural number greater than 0) in descending order of the importance score.

In practical applications, the operation of generating target questions according to target symptom features in step 909 may specifically include: merging target symptom features according to types, and acquiring question entities of corresponding types from the knowledge graph; and generating target questions according to the question entities and the target symptom features of the types.

Specifically, according to at least one target symptom feature of a type, field filling may be performed on the question entity template corresponding to the type, so as to carry information of the at least one target symptom feature in the obtained target questions.

In summary, the inquiry information processing method in the embodiments of this application has the following technical effects:

Firstly, in the embodiments of this application, multiple input modes such as a selection input, a symptom input and a phrase input of an answer option are supported. For a user input of any input mode, the user input can be converted into a symptom feature with a standard description in a knowledge graph by means of a symptom identification method. In this way, the user experience can be improved.

Secondly, in the embodiments of this application, prior to an inquiry to a user, emergency and severe cases can be excluded, and when user symptom features correspond to the emergency and severe cases, the user is advised to seek medical treatment, which can improve the security of the inquiry information processing.

Moreover, in the embodiments of this application, the process of performing disease prediction processing and generating target questions may be a dynamic process. Therefore, the target questions more related to the user disease features can be obtained according to the accumulation of the user disease features in the inquiry process, and the rationality of the inquiry can thus be improved.

Furthermore, according to the inquiry proportion of corresponding symptom features of candidate diseases, the number of inquiry rounds, and the sufficiency of the determination basis of a user symptom set to the candidate diseases in the embodiments of this application, stop conditions are set, and the inquiry is ended when the stop conditions are met, which can not only improve the flexibility of the inquiry, but also reduce the number of inquiry rounds, thereby improving the inquiry efficiency and improving the user experience.

It is to be noted that the foregoing method embodiments are expressed as a series of action combinations for the purpose of brief description, but those skilled in the art may know that because some steps may be performed in other sequences or simultaneously according to the embodiments of this application, the embodiments of this application are not limited to a described action sequence. In addition, those skilled in the art may also know that the embodiments described in this specification are all preferred embodiments, and an action involved is not necessarily mandatory in the embodiments of this application.

### Apparatus Embodiment

Referring to FIG. 10, a structural block diagram of an embodiment of an inquiry information processing apparatus according to this application is shown. The apparatus may specifically include: a user disease feature determination module 1001, a user disease feature processing module 1002 and a question generation module 1003.

The user disease feature determination module 1001 is configured to determine user disease features according to at least one user input.

The user disease feature processing module 1002 is configured to perform disease prediction on the user disease features to obtain corresponding candidate diseases.

The question generation module 1003 is configured to generate target questions according to disease features corresponding to the candidate diseases, the target questions being used for performing an inquiry on a user.

In some embodiments, the user disease feature processing module 1002 may include:
a candidate disease determination module, configured to determine candidate diseases corresponding to the user disease features according to matching information between the user disease features and disease features corresponding to diseases.

In some embodiments, the question generation module 1003 may include:
a question entity acquisition module, configured to acquire corresponding question entities from a knowledge graph according to disease feature entities corresponding to the disease features, the question entities being used for representing questions related to the disease feature entities; and
a first question generation module, configured to generate target questions according to the question entities.

In some embodiments, the question entities may include question entity templates, and the first question generation module may include:
a field filling module, configured to perform field filling on the question entity templates according to the disease features to obtain target questions.

In some embodiments, the field filling module may include:
a first field filling module, configured to perform, according to at least one disease feature of a type, field filling on the question entity template corresponding to the type, so as to carry information of the at least one disease feature in the obtained target questions.

In some embodiments, fields of the question entity templates may include: question text fields and answer option fields.

The field filling module may include:
a second field filling module, configured to fill information of the disease features in the answer option fields of the question entity templates.

In some embodiments, the field filling module may include:
a third field filling module, configured to fill jump relationship fields of the question entity templates according to hit action attributes in the disease feature entities corresponding to the disease features.

In some embodiments, the question entities may include question entity instances, and the question generation module may include:
a second question generation module, configured to acquire target questions from the question entity instances corresponding to the disease features.

In some embodiments, the question entity acquisition module may include:
a first question entity acquisition module, configured to acquire corresponding question entities from the knowledge graph according to hit action attributes in the disease feature entities corresponding to the disease features when answer options corresponding to the disease features are selected; and/or,
a second question entity acquisition module, configured to acquire corresponding question entities from the knowledge graph according to jump relationship fields in question entities corresponding to the disease features when answer options corresponding to the disease features are selected.

In some embodiments, the question generation module 1003 may include:
a target disease feature determination module, configured to determine target disease features from the disease features corresponding to the candidate diseases according to importance scores corresponding to the disease features, the target disease features being configured to represent user disease features inquired in the current inquiry round;
an entity determination module, configured to acquire, according to types corresponding to the target disease features, question entities of the corresponding types from the knowledge graph, the question entities being used for representing questions related to the disease feature entities; and
a third question generation module, configured to generate target questions according to the question entities and the target disease features of the types.

In some embodiments, the at least one user input may include:
an active input; or
an active input and a reply to preset questions; or
an active input and a reply to the target questions; or
an active input and replies to preset questions and target questions.

In some embodiments, the target questions may include question text and answer options.

The at least one user input may include answer options selected by the user.

In some embodiments, the apparatus may further include:
a preset question entity acquisition module, configured to acquire question entities corresponding to preset disease feature entities from the knowledge graph according to the user disease features, the question entities being used for representing questions related to the preset disease feature entities;
a preset disease question determination module, configured to determine preset disease questions according to the question entities, the preset disease questions including: question text and at least one preset option; and
an advice output module, configured to output corresponding medical advice information in response to receiving a selection operation of the user for any preset option.

In some embodiments, the apparatus may further include:
a score determination module, configured to determine scores of the candidate diseases according to probability features.

The probability features may include at least one of the following features:
conditional probabilities of the disease features matching the user disease features under the condition of the candidate diseases;
penalty factors of the disease features matching the user disease features under the condition of the candidate diseases;
incidence probabilities of the candidate diseases in a disease system;
and an incidence probability of the disease system.

In some embodiments, the apparatus may further include:
a stop module, configured to notify the question generation module to stop executing the operation of generating target questions in response to meeting stop conditions upon obtaining the candidate diseases.

In some embodiments, the apparatus may further include:
a processing result output module, configured to output an inquiry information processing result in response to meeting stop conditions upon obtaining the candidate diseases, the inquiry information processing result including: information of the candidate diseases.

In some embodiments, the inquiry information processing result may further include: disease features present to the user and disease features not present to the user.

In some embodiments, the stop conditions may include at least one of the following conditions:
a score of at least one candidate disease is greater than a score threshold;
the difference between scores of multiple candidate diseases meets difference conditions;
an inquiry proportion of the disease features corresponding to the candidate diseases meets a proportion condition; and
the number of inquiry rounds exceeds a round threshold.

In summary, the inquiry information processing apparatus in the embodiments of this application has the following technical effects:

Firstly, in the embodiments of this application, multiple input modes such as a selection input, a symptom input and a phrase input of an answer option are supported. For a user input of any input mode, the user input can be converted into a symptom feature with a standard description in a knowledge graph by means of a symptom identification method. In this way, the user experience can be improved.

Secondly, in the embodiments of this application, prior to an inquiry to a user, emergency and severe cases can be excluded, and when user symptom features correspond to the emergency and severe cases, the user is advised to seek medical treatment, which can improve the security of the inquiry information processing.

Moreover, in the embodiments of this application, the process of performing disease prediction processing and generating target questions may be a dynamic process. Therefore, the target questions more related to the user disease features can be obtained according to the accumulation of the user disease features in the inquiry process, and the rationality of the inquiry can thus be improved.

Furthermore, according to the inquiry proportion of corresponding symptom features of candidate diseases, the number of inquiry rounds, and the sufficiency of the determination basis of a user symptom set to the candidate diseases in the embodiments of this application, stop conditions are set, and the inquiry is ended when the stop conditions are met, which can not only improve the flexibility of the inquiry, but also reduce the number of inquiry rounds, thereby improving the inquiry efficiency and improving the user experience.

An apparatus embodiment is basically similar to a method embodiment, and therefore is described briefly. For related parts, reference may be made to partial descriptions in the method embodiment.

The various embodiments in this specification are all described in a progressive manner. Description of each of the embodiments focuses on differences from other embodiments, and reference may be made to each other for the same or similar parts among respective embodiments.

The specific manners of performing operations by the various modules of the apparatus in the foregoing embodiments are described in detail in the embodiments related to the methods, and are not further described in detail herein.

Embodiments of this application provide an apparatus for processing inquiry information, including a memory, and one or more programs. The one or more programs are stored in the memory and configured to be executed by one or more processors, including instructions for performing the following operations: determining user disease features according to at least one user input; performing disease prediction on the user disease features to obtain corresponding candidate diseases; acquiring question entities from a knowledge graph according to disease feature entities corresponding to the candidate diseases, the question entities being used for representing questions related to the disease feature entities; and generating target questions according to the question entities, the target questions being used for performing an inquiry on a user.

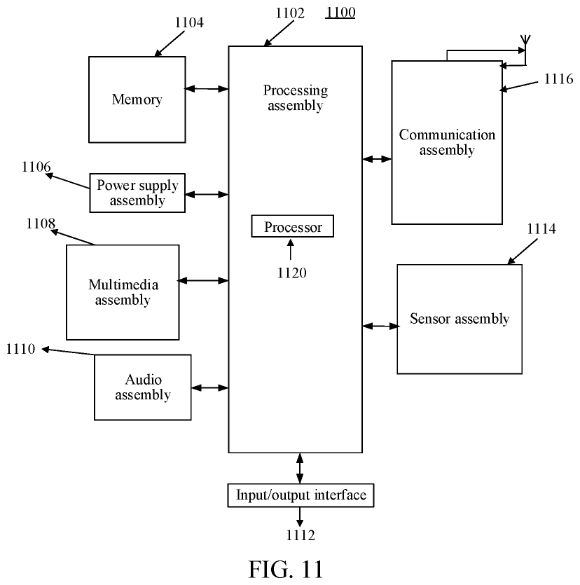

FIG. 11 is a block diagram of an apparatus 1100 for processing inquiry information according to an exemplary embodiment. For example, the apparatus 1100 may be a mobile phone, a computer, a digital broadcasting terminal, a messaging device, a game console, a tablet device, a medical device, a fitness facility, a personal digital assistant, or the like.

Referring to FIG. 11, the apparatus 1100 may include one or more of the following assemblies: a processing assembly 1102, a memory 1104, a power supply assembly 1106, a multimedia assembly 1108, an audio assembly 1110, an input/output (I/O) interface 1112, a sensor assembly 1114, and a communication assembly 1116.

The processing assembly 1102 usually controls the whole operation of the apparatus 1100, such as operations associated with displaying, a phone call, data communication, a camera operation, and a recording operation. The processing assembly 1102 may include one or more processors 1120 to execute instructions, to complete all or some steps of the foregoing method. In addition, the processing assembly 1102 may include one or more modules, to facilitate the interaction between the processing assembly 1102 and other assemblies. For example, the processing assembly 1102 may include a multimedia module, to facilitate the interaction between the multimedia assembly 1108 and the processing assembly 1102.

The memory 1104 is configured to store various types of data to support operations on the apparatus 1100. Examples of the data include instructions, contact data, phonebook data, messages, pictures, videos, and the like of any application or method used to be operated on the apparatus 1100. The memory 1104 may be implemented by any type of volatile or non-volatile storage devices or a combination thereof, such as a static random access memory (SRAM), an electrically erasable programmable read-only memory (EPROM), a programmable read-only memory (PROM), a read-only memory (ROM), a magnetic memory, a flash memory, a magnetic disc, or an optical disc.

The power supply assembly 1106 provides power to various assemblies of the apparatus 1100. The power supply assembly 1106 may include a power supply management system, one or more power supplies, and other assemblies associated with generating, managing and allocating power for the apparatus 1100.

The multimedia assembly 1108 includes a screen providing an output interface between the apparatus 1100 and a user. In some embodiments, the screen may include a liquid crystal display (LCD) and a touch panel (TP). If the screen includes a TP, the screen may be implemented as a touchscreen, to receive an input signal from the user. The TP includes one or more touch sensors to sense touching, sliding, and gestures on the TP. The touch sensor may not only sense the boundary of touching or sliding operations, but also detect duration and pressure related to the touching or sliding operations. In some embodiments, the multimedia assembly 1108 includes a front camera and/or a rear camera. When the apparatus 1100 is in an operation mode, such as a shoot mode or a video mode, the front camera and/or the rear camera may receive external multimedia data. Each front camera and rear camera may be a fixed optical lens system or have a focal length and an optical zooming capability.

The audio assembly 1110 is configured to output and/or input an audio signal. For example, the audio assembly 1110 includes a microphone (MIC), and when the apparatus 1100 is in an operation mode, such as a call mode, a recording mode, and a voice data processing mode, the MIC is configured to receive an external audio signal. The received audio signal may be further stored in the memory 1104 or transmitted via the communication assembly 1116. In some embodiments, the audio assembly 1110 further includes a loudspeaker, configured to output an audio signal.

The I/O interface 1112 provides an interface between the processing assembly 1102 and an external interface module. The external interface module may be a keyboard, a click wheel, buttons, or the like. The buttons may include, but not limited to: a homepage button, a volume button, a start-up button, and a locking button.

The sensor assembly 1114 includes one or more sensors, configured to provide status evaluation in each aspect to the apparatus 1100. For example, the sensor assembly 1114 may detect an opened/closed status of the apparatus 1100, and relative positioning of the assembly. For example, the assembly is a display and a small keyboard of the apparatus 1100. The sensor assembly 1114 may further detect the position change of the apparatus 1100 or one assembly of the apparatus 1100, the existence or nonexistence of contact between the user and the apparatus 1100, the azimuth or acceleration/deceleration of the apparatus 1100, and the temperature change of the apparatus 1100. The sensor assembly 1114 may include a proximity sensor, configured to detect the existence of nearby objects without any physical contact. The sensor assembly 1114 may further include an optical sensor, such as a CMOS or CCD image sensor, that is used in an imaging application. In some embodiments, the sensor assembly 1114 may further include an acceleration sensor, a gyroscope sensor, a magnetic sensor, a pressure sensor, or a temperature sensor.

The communication assembly 1116 is configured to facilitate communication in a wired or wireless manner between the apparatus 1100 and other devices. The apparatus 1100 may access a wireless network based on communication standards, such as WiFi, 2G, or 3G, or a combination thereof. In an exemplary embodiment, the communication assembly 1116 receives a broadcast signal or broadcast related information from an external broadcast management system via a broadcast channel. In an exemplary embodiment, the communication assembly 1116 further includes a near field communication (NFC) module, to promote short range communication. For example, the NFC module may be implemented based on a radio frequency identification (RFID) technology, an infra-red data association (IrDA) technology, an ultra wideband (UWB) technology, a Bluetooth (BT) technology, and other technologies.

In an exemplary embodiment, the apparatus 1100 may be implemented as one or more application specific integrated circuit (ASIC), a digital signal processor (DSP), a digital signal processing device (DSPD), a programmable logic device (PLD), a field programmable gate array (FPGA), a controller, a micro-controller, a microprocessor or other electronic elements, so as to perform the foregoing method.

In an exemplary embodiment, a non-transitory computer readable storage medium including instructions, for example, a memory 1104 including instructions, is further provided, and the foregoing instructions may be executed by a processor 1120 of the apparatus 1100 to complete the foregoing method. For example, the non-transitory computer readable storage medium may be a ROM, a random access memory (RAM), a CD-ROM, a magnetic tape, a floppy disk, an optical data storage device, or the like.

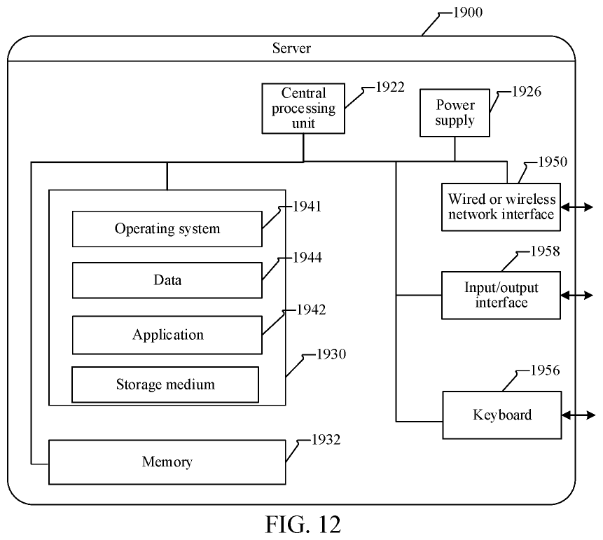

FIG. 12 is a schematic structural diagram of a server according to some embodiments of this application. The server 1900 may vary greatly due to different configurations or performance, and may include one or more central processing units (CPUs) 1922 (for example, one or more processors) and memories 1932, and one or more storage media 1930 (for example, one or more mass storage devices) that store applications 1942 or data 1944. The memory 1932 and the storage medium 1930 may be configured for transient storage or permanent storage. A program stored in the storage medium 1930 may include one or more modules (which are not marked in the figure), and each module may include a series of instruction operations on the server. Further, the CPU 1922 may be configured to communicate with the storage medium 1930, and perform, on the server 1900, the series of instruction operations in the storage medium 1930.

The server 1900 may further include one or more power supplies 1926, one or more wired or wireless network interfaces 1950, one or more input/output interfaces 1958, one or more keyboards 1956, and/or, one or more operating systems 1941, such as Windows Server™, Mac OS X™, Unix™, Linux™, and FreeBSD™.

A non-transitory computer readable storage medium is provided. When instructions in the storage medium is executed by a processor of an apparatus (a server or a terminal), the apparatus is enabled to perform the inquiry information processing method shown in any one of FIGS. 2 to 9.

A non-transitory computer readable storage medium is provided. When instructions in the storage medium is executed by a processor of an apparatus (a server or a terminal), the apparatus is enabled to perform an inquiry information processing method. The method includes: determining user disease features according to at least one user input; performing disease prediction on the user disease features to obtain corresponding candidate diseases; acquiring question entities from a knowledge graph according to disease feature entities corresponding to the candidate diseases, the question entities being used for representing questions related to the disease feature entities; and generating target questions according to the question entities, the target questions being used for performing an inquiry on a user.

After considering the specification and implementing the present disclosure, those skilled in the art may readily think of other implementations of this application. This application is intended to cover any variation, use, or adaptive change of this application. These variations, uses, or adaptive changes follow the general principles of this application and include common general knowledge or common technical means, which are not disclosed in the present disclosure, in the art. The specification and the embodiments are considered as merely exemplary, and the real scope and spirit of this application are pointed out in the following claims.

In this application, the term “unit” or “module” in this application refers to a computer program or part of the computer program that has a predefined function and works together with other related parts to achieve a predefined goal and may be all or partially implemented by using software, hardware (e.g., processing circuitry and/or memory configured to perform the predefined functions), or a combination thereof. Each unit or module can be implemented using one or more processors (or processors and memory). Likewise, a processor (or processors and memory) can be used to implement one or more modules or units. Moreover, each module or unit can be part of an overall module that includes the functionalities of the module or unit. It is to be understood that this application is not limited to the precise structures described above and shown in the accompanying drawings, and various modifications and changes can be made without departing from the scope of this application. The scope of this application is subject only to the appended claims.

The foregoing descriptions are merely preferred embodiments of this application, but are not intended to limit this application. Any modification, equivalent replacement, or improvement made within the spirit and principle of this application shall fall within the protection scope of this application.

The inquiry information processing method, the inquiry information processing apparatus, and the apparatus for processing inquiry information provided in this application are described above in detail. Although the principles and implementations of this application are described by using specific examples herein, the descriptions of the foregoing embodiments are merely intended to help understand the method and the core idea of the method of this application. Meanwhile, those of ordinary skill in the art may make modifications to the specific implementations and application range according to the idea of this application. In conclusion, the content of this specification is not to be construed as a limitation to this application.

What is claimed is:

1. An inquiry information processing method performed at a computing device, the method the method comprising:
determining user disease features according to at least one user input;
performing disease prediction on the user disease features to obtain corresponding candidate diseases;
acquiring question entities from a knowledge graph according to disease feature entities corresponding to the candidate diseases, the question entities being used for representing questions related to the disease feature entities; and
generating target questions according to the question entities, the target questions being used for performing an inquiry on a user that submits the at least one user input.

2. The method according to claim 1, wherein the performing disease prediction on the user disease features comprises:
determining candidate diseases corresponding to the user disease features according to matching information between the user disease features and disease features corresponding to diseases.

3. The method according to claim 1, wherein the question entities comprise question entity templates, and the generating target questions according to the question entities comprises:
performing field filling on the question entity templates according to the disease features of the candidate diseases to obtain target questions.

4. The method according to claim 3, wherein the performing field filling on the question entity templates comprises:
performing, according to at least one disease feature of a type, field filling on the question entity template corresponding to the type, so as to carry information of the at least one disease feature in the obtained target questions.

5. The method according to claim 3, wherein fields of the question entity templates comprise: question text fields and answer option fields;
the performing field filling on the question entity templates comprises:
filling information of the disease features of the candidate diseases in the answer option fields of the question entity templates.

6. The method according to claim 3, wherein the performing field filling on the question entity templates comprises:
filling jump relationship fields of the question entity templates according to hit action attributes in the disease feature entities corresponding to the disease features of the candidate diseases.

7. The method according to claim 1, wherein the question entities comprise question entity instances, the method further comprises:
acquiring target questions from the question entity instances corresponding to the disease features of the candidate diseases.

8. The method according to claim 1, wherein the acquiring question entities from a knowledge graph comprises:
acquiring question entities from the knowledge graph according to hit action attributes in the disease feature entities corresponding to the disease features of the candidate diseases when answer options corresponding to the disease features of the candidate diseases are selected; and/or,
acquiring question entities from the knowledge graph according to jump relationship fields in question entities corresponding to the disease features when answer options corresponding to the disease features of the candidate diseases are selected.

9. The method according to claim 1, wherein the acquiring question entities from a knowledge graph comprises:
determining target disease features from the disease features corresponding to the candidate diseases according to importance scores corresponding to the disease features, the target disease features being used for representing user disease features inquired in the current inquiry round;
acquiring, according to types corresponding to the target disease features, question entities of the corresponding types from the knowledge graph, the question entities being used for representing questions related to the disease feature entities; and
the generating target questions comprises:
generating target questions according to the question entities and the target disease features of the types.

10. A computing device, comprising a processor and a memory, the memory storing instructions that, when executed by the processor, cause the computing device to perform an inquiry information processing method including:
determining user disease features according to at least one user input;
performing disease prediction on the user disease features to obtain corresponding candidate diseases;
acquiring question entities from a knowledge graph according to disease feature entities corresponding to the candidate diseases, the question entities being used for representing questions related to the disease feature entities; and
generating target questions according to the question entities, the target questions being used for performing an inquiry on a user that submits the at least one user input.

11. The computing device according to claim 10, wherein the performing disease prediction on the user disease features comprises:
determining candidate diseases corresponding to the user disease features according to matching information between the user disease features and disease features corresponding to diseases.

12. The computing device according to claim 10, wherein the question entities comprise question entity templates, and the generating target questions according to the question entities comprises:
performing field filling on the question entity templates according to the disease features of the candidate diseases to obtain target questions.

13. The computing device according to claim 12, wherein the performing field filling on the question entity templates comprises:
performing, according to at least one disease feature of a type, field filling on the question entity template corresponding to the type, so as to carry information of the at least one disease feature in the obtained target questions.

14. The computing device according to claim 12, wherein fields of the question entity templates comprise: question text fields and answer option fields;
the performing field filling on the question entity templates comprises:
filling information of the disease features of the candidate diseases in the answer option fields of the question entity templates.

15. The computing device according to claim 12, wherein the performing field filling on the question entity templates comprises:
filling jump relationship fields of the question entity templates according to hit action attributes in the disease feature entities corresponding to the disease features of the candidate diseases.

16. The computing device according to claim 9, wherein the question entities comprise question entity instances, the method further comprises:
acquiring target questions from the question entity instances corresponding to the disease features of the candidate diseases.

17. The computing device according to claim 9, wherein the acquiring question entities from a knowledge graph comprises:
acquiring question entities from the knowledge graph according to hit action attributes in the disease feature entities corresponding to the disease features of the candidate diseases when answer options corresponding to the disease features of the candidate diseases are selected; and/or,
acquiring question entities from the knowledge graph according to jump relationship fields in question entities corresponding to the disease features when answer options corresponding to the disease features of the candidate diseases are selected.

18. The computing device according to claim 9, wherein the acquiring question entities from a knowledge graph comprises:
determining target disease features from the disease features corresponding to the candidate diseases according to importance scores corresponding to the disease features, the target disease features being used for representing user disease features inquired in the current inquiry round;
acquiring, according to types corresponding to the target disease features, question entities of the corresponding types from the knowledge graph, the question entities being used for representing questions related to the disease feature entities; and
the generating target questions comprises:
generating target questions according to the question entities and the target disease features of the types.

19. A non-transitory computer-readable storage medium, comprising instructions that, when executed by a processor of a computing device, cause the computing device to perform an inquiry information processing method including:
determining user disease features according to at least one user input;
performing disease prediction on the user disease features to obtain corresponding candidate diseases;
acquiring question entities from a knowledge graph according to disease feature entities corresponding to the candidate diseases, the question entities being used for representing questions related to the disease feature entities; and
generating target questions according to the question entities, the target questions being used for performing an inquiry on a user that submits the at least one user input.

20. The non-transitory computer-readable storage medium according to claim 19, wherein the question entities comprise question entity templates, and the generating target questions according to the question entities comprises:
performing field filling on the question entity templates according to the disease features of the candidate diseases to obtain target questions.
